(19) United States
(12) Patent Application Publication
(10) Pub. No.: US 2026/0183003 A1
(43) Pub. Date: Jul. 2, 2026

(54) INTRAVASCULAR LITHOTRIPSY DEVICES WITH THIN-WALLED BALLOONS AND SYSTEMS

(71) Applicant: Cardiovascular Systems, Inc., St. Paul, MN (US)

(72) Inventor: Joseph P. Higgins, Minnetonka, MN (US)

(21) Appl. No.: 19/129,816

(22) PCT Filed: Nov. 14, 2023
(86) PCT No.: PCT/US2023/037234

§ 371 Date: May 14, 2025

Related U.S. Application Data
(60) Provisional application No. 63/425,169, filed on Nov. 14, 2022.

(51) Int. Cl. A61B 17/22; A61L 29/14; A61M 25/10

(52) U.S. Cl. CPC A61B 17/22022; A61L 29/14; A61B 2017/22025; A61B 2017/22038; A61B 2017/22062; A61M 2025/1043

(57) ABSTRACT

Improved IVL systems and components having a reduced tendency for failure caused by undesirable electrical arcing and/or current flow and/or plasma discharge between IVL emitters and a deployed balloon. Additionally, or alternatively, the present invention provides improved IVL systems and components having a reduced tendency for failure caused by possibility of undesirable electrical discharge and/or electrical arcing and/or current flow between the deployed balloon and tissue. This undesirable flow of energy may occur during therapy delivery but may also occur during pauses or at the end of therapy pulses due to a prolonged charging of the fluid solution in the balloon and its effects outside of the balloon.

### CROSS-REFERENCE TO RELATED APPLICATIONS

This application claims priority to and the benefit of U.S. Provisional Application No. 63/425,169, filed Nov. 14, 2022, and entitled INTRAVASCULAR LITHOTRIPSY DEVICES AND SYSTEM, the entire contents of which are incorporated herein by reference in their entireties.

### TECHNICAL FIELD

Intravascular lithotripsy catheters for use in intravascular medical procedures.

### BACKGROUND OF THE INVENTION

Calcification of blood vessels hinders proper blood flow and is associated with adverse health consequences including severe blood flow restriction. Calcification can range from minor to severe and calcification patterns can also vary significantly.

One successful approach to alleviating adverse effects of calcification is orbital atherectomy. While highly successful and considered the gold standard for treating difficult calcification challenges, other options are available to physicians for less challenging calcification patterns.

Intravascular lithotripsy (IVL) devices are available for some calcification patterns. Disposable IVL balloon devices are provided in different designs and sizes for peripheral or coronary indications. All designs utilize a reusable power source such as an IVL generator. One reusable generator comprises the following specification:

| | |
|---|---|
| Power | 110-240 VAC; 50-60 Hz; Single Phase, 15 A |
| | service |
| Size | 11″ (28.0 cm) high × 6″ (15.2 cm) wide × |
| | 11.5″ (29.2 cm) deep |
| Weight | 15 pounds (6.8 kg) |
| Output | Proprietary pulse delivery system. Output |
| | voltage 3000 volts peak, pulse |
| | frequency 1 Hz |
| Mobility | Product is designed to be mounted to an IV |
| | pole |
| Length | 5 ft (1.53 m) |
| Compatibility | Male key distally designed to connect only |
| | to catheter. |
| Operation | Lithotripsy pulsing is activated by pushing a |
| | button on the Connector Cable. |
| Use | Re-usable |

Intravascular lithotripsy (IVL) devices are available for some calcification patterns. Disposable IVL balloon devices are provided in different designs and sizes for peripheral or coronary indications. All designs utilize a reusable power source such as an IVL generator. One reusable generator comprises the following specification:

One such disposable device consists of a 0.014-inch guidewire-compatible, fluid-filled balloon catheter with two lithotripsy emitters incorporated into the shaft of the 12-mm-long balloon segment. A fluid filled balloon (e.g. a 50/50 saline contrast medium) is inflated to about 4 atm and then electrical pulses are provided to the emitters that create high voltage sparks to provide the therapy. Acoustic waves are created and the calcium is fractured.

Reported details of a known IVL balloon include features as illustrated in FIG. 9. Specifically, an example working length of such an IVL catheter balloon zone is defined by marker bands between which emitters are positioned at spaced locations. Different spacing can be provided between a pair of emitters as well as spacing from the emitters to the maker bands. Also illustrated is an energy profile of the balloon that is shown as a smooth energy profile over a center portion of the balloon. A manner of arrangement of the emitters to create an optimized overlap zone is also illustrated.

Balloon life and durability are known problems associated with currently available IVL devices. Maximum pulse counts are associated with particular designs, with one device limiting the pulses to 300 pulses and another product limiting pulse counts to 160 per balloon, even less per electrode. After a number of pulses far below the limits, deleterious material may be found within the saline filled balloon that is caused by the electrical pulses and generated energy (bubble creation, collapse, heat, shockwaves). The number of pulses is controlled. In one example, the maximum number of continuous pulses is 30, and the minimum pause time is 10 seconds.

Balloons for IVL systems are constructed from tough materials such as Nylon or PEBAX. One such system was known as the S4 system available from Shockwave Medical of Santa Clara, California. The manufacturer initially had a much thinner balloon in its system; however, it failed in use on humans. The manufacturer recalled the S4 system and stated the recall of the S4 catheters was based on an inability of the balloon to maintain inflation due to suboptimal balloon wall thickness in at least some of the sizes. It is believed that the current thickness of the balloon in the S4 system has approximately doubled to approximately 0.0009 inches.

Efforts have been made to increase balloon integrity in an IVL system. See for example, U.S. Pat. Application Publication No. 2021-0378743 that describes balloon integrity protection components.

Balloon life and durability are important to a physician as if therapy is not complete after pulse count limits have been reached, the balloon must be removed, detached from the power supply and an entirely new disposable balloon must be threaded through the patient's vasculature to complete the therapy. This is undesirable for a variety of reasons including time, cost, balloon locating/overlap, and further exposure of the patient to possible harm.

The precise mechanism of balloon failure in IVL devices is not widely known. Theories and possible culprits abound. The energy of a plasma spark, cavitation bubbles and heat are known agents with highly destructive qualities.

### SUMMARY OF THE INVENTION

While not bound to any one theory, the present invention is directed to the possibility of failure due at least in part to electrical arcing and/or current flow and/or plasma discharge between IVL emitters and a deployed balloon or the possibility of undesirable electrical discharge and/or electrical arcing and/or current flow between the deployed balloon and tissue.

The present invention provides improved IVL systems and components having a reduced tendency for failure caused by undesirable electrical arcing and/or current flow and/or plasma discharge between IVL emitters and a deployed balloon. Additionally, or alternatively, the present invention provides improved IVL systems and components having a reduced tendency for failure caused by possibility of undesirable electrical discharge and/or electrical arcing and/or current flow between the deployed balloon and tissue. This undesirable flow of energy may occur during therapy delivery but may also occur during pauses or at the end of therapy pulses due to a prolonged charging of the fluid solution in the balloon and its effects outside of the balloon.

The disclosed invention addresses shortcomings in current intravascular lithotripsy systems, by preventing or discouraging the undesirable discharge of energy described above that may cause accidental injury to the patient or device itself, especially the balloon. The present invention is especially desirable in cardiac applications where the unintended discharge of electrical energy to a patient may harm or cause damage to the patient's cardiac cycle, conduction path and/or its ability to properly control the heart. The invention includes modification of existing devices to insulate, isolate or shield the balloon (by for example, adding electrical insulation), thereby preventing or discouraging the unwanted release of energy.

The various inventions disclosed herein address these, inter alia, issues.

### BRIEF DESCRIPTION OF THE DRAWINGS

These drawings are exemplary illustrations of certain embodiments and, as such, are not intended to limit the disclosure.

FIG. 1 illustrates a system for providing intravascular lithotripsy according to an aspect of the present invention.

FIG. 2 illustrates an inflated balloon in a vessel for providing intravascular lithotripsy according to an aspect the present invention.

FIG. 3 illustrates an example of a balloon material according to the present invention.

FIG. 4 illustrates a second example of a balloon material according to the present invention.

FIG. 5 illustrates a third example of a balloon material according to the present invention.

FIG. 6 illustrates a fourth example of a balloon material according to the present invention.

FIG. 7 is a schematic view of another embodiment of the present invention utilizing five electrode pairs.

FIG. 8 is a schematic view of another embodiment showing the wiring of electrode pairs with at least one latching switch.

FIG. 9 is an illustration of features of certain IVL catheters showing details of a working length of such an IVL catheter, an energy profile of the IVL catheter, and the provision of an optimized overlap zone within such an IVL catheter design.

### DETAILED DESCRIPTION

Unless the context appears otherwise, the following terms or phrases shall have the following meaning (in both this singular and plural):

The terms “insulator”, “insulation” or “insulating” mean electrically insulating, and refer to dielectric materials that permit little, if any, flow of electrical current through such material. Insulating materials may also be thermally insulating but are not necessarily so. Materials such as glass, metal oxides, porcelain, paper, plastics, polymers, and rubbers are representative of insulating materials.

The terms “conductor”, conductive” and “conducting” mean electrically conductive, and refer to materials that easily permit the flow of electrical current through such material. Conductive materials may, in some examples, be thermally conductive, but are not always so. Materials such as carbon black, gold, and metals are representative conductive materials.

The phrase “conductive path” refers to a viable path for the flow of electrical current along such path and may, for example, refer to an entire surface of a balloon on component thereof coated with a conductive material, or only a portion of a surface coated with a conductive material such as conductive stripes or predetermined (e.g. rectangular) shapes on the surface or layer of a balloon. The conductive path may be placed on the surface of a balloon via known ink printing or other techniques. Examples of ink printed medical devices are described in U.S. Pat. Nos. 5,836,874; 7,379,767; 9,7636,24; 9,913,594 and 10,751,000 In some instances, such a conductive path may create a virtual or actual faraday cage effect such that unwanted energy flow from the balloon to the patient is prevented.

The phrase “intravascular lithotripsy device” includes devices that create acoustic waves by arcing discharges between electrode components but may also include devices that create acoustic energy within the balloon via laser energy sources. Examples of such laser systems are described in U.S. Pat. Nos. 11,058,492 and 11,246,569 (the entire contents of which are incorporated by reference). Examples of electrically induced systems are described in U.S. Pat. Nos. 8,728,091, 9,642,673 and 10,850,078 and Published U.S. Pat. Appl. No. 2022-0054194 (the entire contents of which are incorporated by reference).

The phrase “lithotripsy emitters” means electrode-based systems and may also include laser or optical systems.

The phrase “high voltage pulse” means an electrical pulse comprising a minimum voltage of at least 2000 volts, and in some embodiments a maximum voltage of at least 3000 volts.

The phrase “thin-walled balloon material” means any compliant, semi-compliant and non-compliant balloon materials that are less than about 0.0009 inches in thickness. Preferably, the thin-walled balloon material comprises material that is less than 0.0009 inches thick in an unstretched state prior to inflation. One such material comprises a polyamide having repeating units linked by amide links. Some Nylon and PEBAX materials are suitable. Such materials may also include materials such as a composite or multilayer structure. Compliant materials include silicone, polyurethane or nitinol materials. In some embodiments, thin-walled balloon materials may comprise balloon in balloon designs and the fluids filling the inner balloon may be dissimilar to the fluid filling the outer balloon to exploit differences in electrical insulative properties, speeds of sound of materials. Compliant, semi-compliant, and non-compliant materials may include nylon, polyurethanes, silicones, polyethylene terephthalate (PET) and other biocompatible materials. In one embodiment, a burst pressure of thin-walled balloons is typically between about 4 and 20 atm, more preferably between 8 and 12 atm and one example is a Nylon 12 material with a rated burst pressure of between about 9 and 12 atm.

The phrase “self-sealing material” means a flowable material capable of flowing into a pinhole in the balloon caused by operation of the system 10.

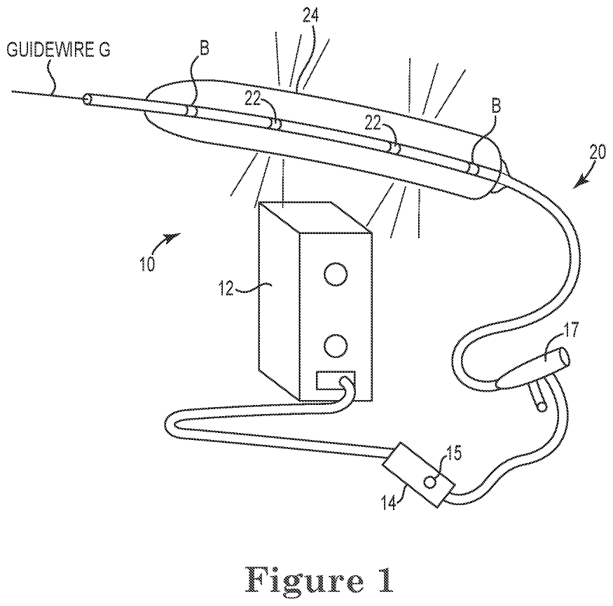

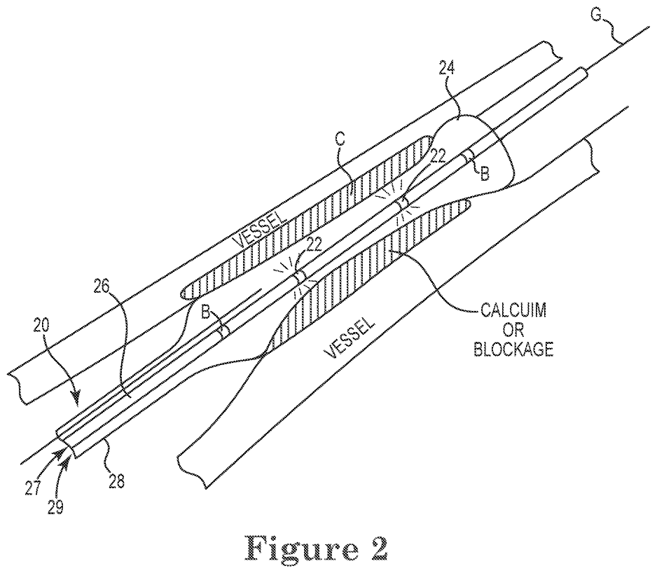

With reference to the Figures, FIGS. 1 and 2 show a system 10 according to the present invention comprising a power source 12 (in the form of an electrical generator, but alternatively in the form of a laser system), a handle 14 with therapy delivery control 15 and a catheter 20 with two lithotripsy emitters 22 (shown in the form of a pair of arcing electrodes, but alternatively they could comprise optical or laser emitters), and a fluid filled balloon 24. Optional marker bands B may be provided. The catheter 20 preferably includes a central tube 26 defining a guide wire lumen 27 through which a guide wire G passes for delivering the balloon 24 at the desired location along the guide wire G. A sheath 28 surrounds the central tube 26 and defines a delivery lumen 29 through which saline can be controllably delivered for balloon 24 inflation. The lumen 29 provides a concentric space around the central tube 26 within with electrode wires (not shown) can be run from the control 15 to the emitters 22 among other components in accordance with the present invention and discussed below. The sheath 28 is connected at a proximal end to a hub 17 that can include any number of ports allowing electrode wires to pass into the lumen 29 along with saline for inflation, the guide wire G, and any number of other components as desired.

The balloon 24 may be placed in a deflated position so as to more readily pass through a patient's vasculature to arrive at the scene of calcification. In use, the balloon 24 will be inflated to a common pressure for angioplasty procedures (e.g. 4 atm) and the therapy actuated via the delivery control 15.

FIG. 2 shows the balloon 24 inflated to a therapy delivery state where the lithotripsy emitters 24 may be “fired” to disrupt the calcium C. Optional indicator bands B may be provided to afford visualization and proper positioning by use of known imaging techniques. The balloon 24 is inflated to a typical angioplasty pressure (e.g. 4 atm) and therapy is delivered. The balloon 24 may naturally expand during or just after the therapy is delivered to clear the vessel for passage of blood.

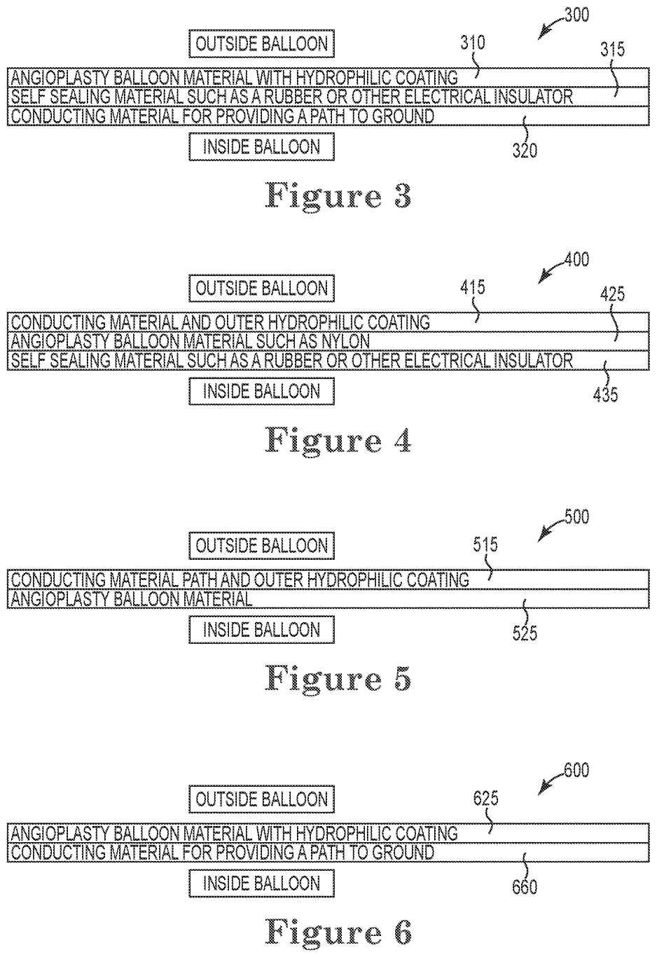

The material of the balloon 24 is inflated well below its burst pressure (e.g. 10 atm) to avoid balloon failure. FIGS. 3-6 describe different embodiments of balloon materials 300, 400, 500 and 600 according to the present invention.

The materials 300, 400, 500 and 600 should not prevent the therapy from the lithotripsy emitters from reaching the calcium. For lithotripsy emitters that emit an acoustic wave at the speed of sound, the materials 300, 400, 500 and 600 should not unduly interfere with the acoustic energy delivered to the calcium. An “acoustic wave” and the speed of wave propagation (called the sound speed) is a material property of the medium. For air it is about 340 m/s and in water and most soft tissues in the body, it is about 1500 m/s. The difference between the sound speed of the materials 300, 400, 500 and 600 and its thickness should not be so significantly different than the speed of the wave in the fluid portion of the balloon or typically encountered surrounding soft tissues. Typically, the balloon material will result in a balloon diameter between 1.5 mm and 6 mm at about 4 atm, more preferably between 2 and 4 mm, and more preferably about 3.5 mm. Other sizes of the balloon are within the scope of the present invention.

FIG. 3 illustrates an embodiment of material 300 for the balloon 24 that comprises a thin-walled polyamide, comprising repeating units linked by amide links such as nylon 310 with an optional hydrophilic coating to render the device slippery. The material 310 may optionally contain a layer of self-sealing material 315 such as rubber (or other hemostatic material given vicinity to blood), that may flow into a pin hole that the system 10 creates in the balloon during use. The self-sealing material may flow into the pinhole to deter leakage. The material 300 includes a conducting material 320 for providing a path to ground to deter damaging electrical flow out of the balloon to the tissue. The material 300 may afford a balloon 24 whose outer potential is less than the electrical potential of the inner components of the balloon 24.

FIG. 4 illustrates an embodiment of material 400 for the balloon 24 that comprises a conductive material 415 for providing an electrical path for current flow to ground and away from tissue to that comprises a material 425 such as nylon. The conductive material 415 may include an outer hydrophilic coating to render the material 400 slippery. Conductive material 415 deters damaging electrical flow from the balloon to tissue by giving such energy an alternative safe path to ground. The material 400 may include an optional self-sealing material 435 in the inner side of the balloon material 400. The material 400 may optionally provide a balloon catheter whose electrical potential is greater than the internal components of the balloon 400.

In another embodiment, the balloon comprises conductive material on, over, or within the outer wall of the balloon. Construction using materials and technologies commonly found in electroactive polymer constructions, which utilize high voltages in an expected range of use in an IVL device, could be employed in this construction. The coating/embedding should be selected to include conductivity and load carrying capacity which allows for partial to full coupling and discharge of energy delivered into the balloon core/energy delivered. This material should also allow for charging to create a charge differential to resist the charge potential of the electrode from reaching that surface.

FIG. 5 illustrates an alternative embodiment of material 500 for the balloon 24 that comprises a conductive material 515 for providing an electrical path for current flow to ground and away from tissue. The material 400 may also comprise a typical angioplasty material 525 such as nylon. The conductive material 515 deters damaging electrical flow from the balloon to tissue by providing an alternative safe path for such energy to ground.

Alternatively, the conductive paths or materials 415 or 515 may be used to charge the outer surface of the balloon to reduce potential energy to transfer from the inner lumen to the outside of balloon. The charge may be sufficient for some flow to tissue, but not enough to harm the tissue. This helps reduce the changes that a large energy will jump from the balloon 24 to the patient by reducing the charge potential between the emitter and the ground plane of either the patient or the balloon, such that the catheter (current primary electrical circuit design path) ground plane is path of least resistance and impedance to the balloon or the body is increased.

FIG. 6 illustrates an alternative embodiment of material 600 for the balloon 24 that comprises a material 625 such as nylon. The material 600 includes a conductive material 660 within the interior of the balloon 24 to provide a safe path for energy to flow to ground rather from the balloon to the patient. The material 600 may afford a balloon 24 whose outer potential is less than the electrical potential of the inner components of the balloon 24.

Commercially available IVL devices operate at a voltage of slightly less than 3000 volts and include current of between 20 and 300 amps. The present invention may afford the use of higher voltages and/or currents or power while safely protecting the patient.

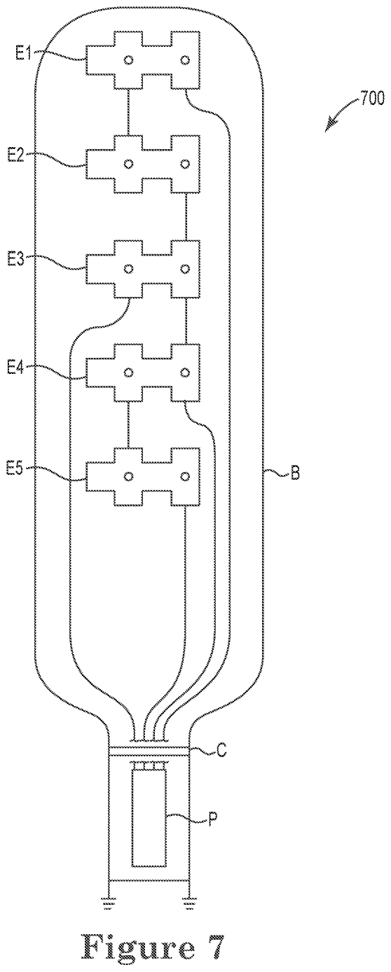

FIG. 7 shows another embodiment of the present invention for an IVL system 700 comprising a fluid fillable balloon B, a catheter (shown schematically as a box C) five electrode pairs E1, E2, E3, E4 and E5 and a power and control source P for the electrode pairs. In this embodiment, the Balloon B, and catheter/delivery tube C is constructed to be capable of acting as a primary or secondary ground/return path for energy from source P that is not partially or fully discharged/grounded to the return path within the balloon.

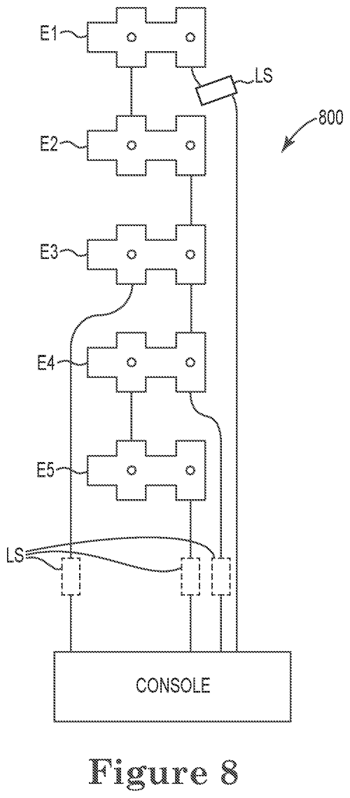

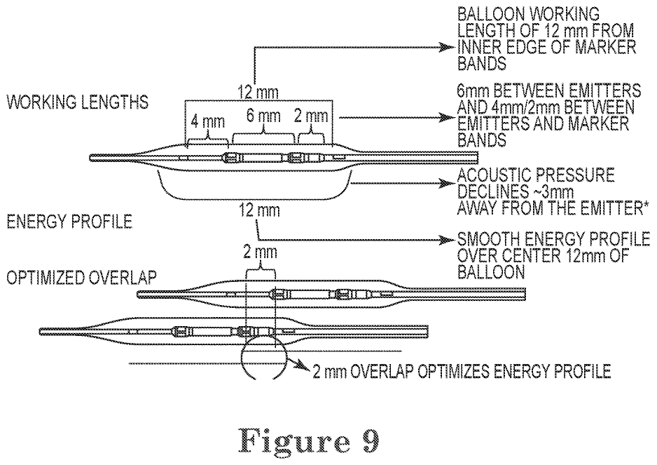

FIG. 8 shows another embodiment of the present invention in a system with, for example, five electrode pairs E1-E5. The system 800 includes at least one heat activated latching switch L1 preferably in the distal portion of the balloon interior space. The latching switch L1 may be triggered either with each activation via balloon/spark heat, and/or generated only when the balloon or fluid within it is heated over a safety threshold at which time the console would identify and latch the signal to allow the console to identify the “short” in the balloon. Once a safe temperature is reached this “circuit” would become functional once again. Various transistor technologies may be of usefulness in this area, and/or the use of temperature activated materials common to this space, such as nitinol (NITI) to activate and reactivate.

In other embodiment, the balloon may have a conductive cage, or even single wire embedded on the inner lumen of the balloon which expands to a diameter of the balloon with very small conductors which have limited impact on the profile and/or deliverability of the balloon and/or catheter.

For electrical isolation of 3000V (roughly IVL voltage) one would need a balloon thickness upwards of 0.005″ dry. So more than 5× thickness. Such a material would have a dielectric strength of 600 v/mil, which is the case with certain specific nylons that are usable in accordance with the present invention.

In one aspect, a preferable balloon wall will have a dielectric strength of 3333 V/mil for a 0.0009″ wall thickness. Such a preferred balloon will have a potential energy and dielectric strength combined to reduce risk to the patient.

Dielectric properties of the material used for making the balloon can also enhance a thin-walled balloon to prevent electrical discharge through or by the balloon to the body of a patient. It is understood that in prior art systems balloons of Nylon (From the Grilamid and Pebax families) and similar materials at thin-wall thickness at 0.0009 can fail during usage, and such failure is believed to be based at least in part of electrical discharge or caused by undesirable electrical arcing and/or current flow and/or plasma discharge between IVL emitters and a deployed balloon to tissue of a patient. So, by making a balloon of a suitable compliant material that has better dielectric properties that are more electrically insulative than Nylon will result in an improved thin-walled balloon. Materials for additional options for balloon materials to increase dielectric properties and dielectric strength include Mylar or Kapton with a higher dielectric resistance. A higher dielectric strength represents a better quality of insulator.

The measurement of dielectric properties involves measurements of a relative permittivity (εᵣ) and a relative permeability (μᵣ) of the material in question. Part of the determination of relative permittivity (εᵣ) is known as the dielectric constant, which is a measure of the amount of energy from an external electrical field that is applied across the material and that is stored in the material. Another part of the relative permittivity (εᵣ) is a loss factor that is a measurement of energy loss from the material due to the external electrical field. For Nylon within an electrical field at a frequency of 2.45 GHz. at room temperature, the dielectric constant is 2.4 with a loss factor of 0.0083. Relative permeability is a similar concept to relative permittivity except that the energy stored and dissipated is magnetic field energy as opposed to electrical field energy. See the publication “Measurement of Dielectric Material Properties, Rohde & Schwarz, 04-2012, hereinafter “R&S publication”, the entire contents of which is hereby incorporated by reference for all purposes. It is also understood that ASTM D149 can be used for determining dielectric strength.

Measurement of the relative permittivity includes a number of methods including a transmission/reflection line method, an open-ended coaxial probe method, a free space method, and a resonant method. Of these methods, the free space method is particularly applicable to measuring relative permittivity of materials that might be suitable for thin-walled balloon making as such materials can be provided as large flat solid materials for testing. It is contemplated that any of the other methods disclosed in thee U&S publication may otherwise be used, wherein the transmission/reflection line method is also very suitable for smaller solid materials

Specifically, for the free space method, the material to be tested is relatively large and flat and situated between two antennas facing each other with the antennas connected to a network analyzer. S parameters are measured by placing a holder midway between the two antennas operating at a set frequency and then placing the material in the holder and measuring the s-parameter again. The influence of the sample holder is cancelled out so that the s-parameter of the material can be determined. The dielectric properties of the material can be determined by post processing the measured reflection and transmission coefficient using known conversion programming that can be provided within the network analyzer or otherwise along with any number of microprocessors, memory, and other computing hardware.

Such a free space method can be performed initially on a material such as Nylon with a thickness of 0.0009 following by performing the same test on other materials of similar thickness. Then, the dielectric properties of various materials can be compared. A suitable material for an expandible balloon as described above and with superior dielectric properties as compared with Nylon will create an improved balloon that is less likely to fail during usage based at least in part of electrical discharge or caused by undesirable electrical arcing and/or current flow and/or plasma discharge between IVL emitters and a deployed balloon to tissue of a patient. The improved thin-walled balloon will be less likely to fail or create electrical discharge or cause undesirable electrical arcing and/or current flow and/or plasma discharge between IVL emitters and a deployed balloon to tissue of a patient.

In another aspect, the present invention may include utilization of a martensitic band/electrode material to allow for the connection of materials eroded from the band and changing the conductivity and ground path through magnetic means (used to collect such eroded materials) to reduce the uncontrolled and variable conductivity of the solution/fluid surrounding the electrodes.

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1. A catheter for intravascular lithotripsy procedures comprising:
an elongated body sized to fit within a blood vessel,
a guide wire lumen extending through the catheter,
a thin-walled balloon located near a distal end of the elongated body with a distal and proximal end of the balloon being sealed to afford inflation and deflation thereof by a fluid;
at least two electrode pairs within the balloon capable of receiving high voltage pulses and generating arcs within the fluid when the fluid inflates the thin-walled balloon, the catheter providing energy within the balloon that is sufficient to create a plasma arc within the fluid resulting in a shock wave within the balloon, and wherein the balloon material is more dielectric than nylon based upon having a relative permittivity that is less than that for nylon.

2. The catheter of claim 1, wherein the balloon material also has a dielectric constant that is lower than that of nylon.

3. The catheter of claim 1, wherein the balloon material has a dielectric strength that is higher than nylon.

4. The catheter of claim 2, wherein the balloon material also has a loss factor that is lower than that of nylon.

5. The catheter of claim 1, wherein the balloon material is 0.0009 inches in thickness or less.

6. The catheter of claim 1, wherein the balloon material has a burst strength of between about 4 and 20 atmospheres.

7. The catheter of claim 1, wherein the thin-walled balloon comprises material that is less than 0.0009 inches thick prior to inflation.

8. A catheter for intravascular lithotripsy procedures comprising:
an elongated body sized to fit within a blood vessel,
a guide wire lumen extending through the catheter,
a thin-walled balloon located near a distal end of the elongated body with a distal and proximal end of the balloon being sealed to afford inflation and deflation thereof by a fluid;
at least two electrode pairs within the balloon capable of receiving high voltage pulses and generating arcs within the fluid when the fluid inflates the thin-walled balloon, the catheter providing energy within the balloon that is sufficient to create a plasma arc within the fluid resulting in a shock wave within the balloon, and wherein the balloon material is thinner than 0.0009 inches thick prior to inflation.

9. The catheter of claim 8, wherein the thin-walled balloon has a relative permittivity that is at least the same as nylon of the Grilamid or Pebax families or less.

10. The catheter of claim 8, wherein the thin-walled balloon has a dielectric constant that is the same or lower than nylon of the Grilamid or Pebax families.

11. The catheter of claim 8, wherein the balloon material has a dielectric strength that is equal to or higher than nylon of the Grilamid or Pebax families.

12. The catheter of claim 10, wherein the balloon material also has a loss factor that is equal to or lower than that of nylon.

13. A catheter for intravascular lithotripsy procedures comprising:
an elongated body sized to fit within a blood vessel,
a guide wire lumen extending through the catheter,
a thin-walled balloon located near a distal end of the elongated body with a distal and proximal end of the balloon being sealed to afford inflation and deflation thereof by a fluid;
at least two electrode pairs within the balloon capable of receiving high voltage pulses and generating arcs within the fluid when the fluid inflates the thin-walled balloon, the catheter providing energy within the balloon that is sufficient to create a plasma arc within the fluid resulting in a shock wave within the balloon, and wherein the balloon material is more dielectric than nylon based upon having a relative permittivity that is less than that for nylon, and wherein the balloon material is thinner than 0.0009 inches thick prior to inflation.

14. The catheter of claim 13, wherein the balloon material also has a dielectric constant that is lower than that of nylon.

15. The catheter of claim 13, wherein the balloon material has a dielectric strength that is higher than nylon.

16. The catheter of claim 14, wherein the balloon material also has a loss factor that is lower than that of nylon.

17. The catheter of claim 13, wherein the balloon material has a burst strength of between about 4 and 20 atmospheres.
